(19) United States
(12) Patent Application Publication
(10) Pub. No.: US 2010/0278758 A1
(43) Pub. Date: Nov. 4, 2010

(54) GEL FOR TEETH TISSUES REMINERALIZATION

(75) Inventors: Tamazi Omarovich Manasherov, Moscow (RU); Svetlana Konstantinovna Matelo, Moskovskaya obl. (RU); Tatjyana Vladimirovna Kupec, Moskovskaya obl. (RU)

Correspondence Address: BROOKS KUSHMAN P.C., 1000 TOWN CENTER, TWENTY-SECOND FLOOR, SOUTHFIELD, MI, 48075, US

(73) Assignee: OBTHESTVO S OGRANICHENNOYJ OTVETSTVENNOSTJYU "WDS", Moscow (RU)

(21) Appl. No.: 12/812,064

(22) PCT Filed: Jan. 21, 2009
(86) PCT No.: PCT/RU2009/000011

§ 371 Date: Jul. 8, 2010

(30) Foreign Application Priority Data
Feb. 7, 2008 (EA) 200800988

(51) Int. Cl. A61K 8/55; A61Q 11/00

(52) U.S. Cl. 424/57

(57) ABSTRACT

Technical result of the invention is achieved by practical use of effective remineralizing formulation containing accessible components, which can be used by a patient without any help for caries prophylaxis, non-carious affections treatment, teeth hypersensitivity treatment and teeth visual appearance (color and gloss) improvement. The problem specified is solvable by preparing gel comprising xylitol 2-25 wt. %, calcium glycerophosphate 0.1-3.0 wt. %, magnesium ion source 0.01-0.50 wt. % and guar gum 0.03-0.30 wt. % as active components and inert components as well used for teeth gels preparation, herein as magnesium ion source there is used magnesium chloride, magnesium sulfate or magnesium nitrate.

### FIELD OF APPLICATION

The invention relates to stomatology and perfume industry, in particular to gel for teeth tissues remineralization.

### BACKGROUND OF INVENTION

There is known formulation intended for teeth tissues remineralization containing calcium gluconate, cellulose gum, dimethylsulfoxide, furacilin, glycerine and water (SU A3 No 1774870, IPC 5 A61K 6/02, 1992).

The prepared formulation is dried and then used in the form of elastic film for application on injured parts of teeth with the surface cleaned from teeth deposits before. The applied film is moistened by saliva, transformed into gel and resolved step by step for 60-120 minutes, thus providing for the prolonged coming out of remineralizing substance and its extended effect on solid teeth tissues. The necessity of film appliance and duration of resolution make it difficult for a patient to use the formulation at home.

### SUBSTANCE OF INVENTION

Technical result of the invention is achieved by practical use of effective remineralizing formulation containing accessible components, which can be used by a patient without any help for caries prophylaxis, non-carious affections treatment, teeth hypersensitivity treatment and teeth visual appearance (color and gloss) improvement.

The technical result can be enhanced by gel for teeth tissues remineralization which comprises xylitol 2-25 wt %, calcium glycerophosphate 0.1-3.0 wt %, magnesium ion source 0.01-0.50 wt. % and guar gum 0.03-0.30 wt. % as active components and inert components as well used for teeth gels preparation.

As magnesium ion source there is used magnesium salt which dissociates into ions completely or partially, particularly there is used one of the following salts: magnesium chloride, magnesium sulfate or magnesium nitrate.

Xylitol in the formulation performs several functions: being a sweetener, xylitol improves its taste properties; being a polyatomic alcohol, it acts as a water-retaining component. Mechanism of its involvement in biochemical metabolism of streptococci is characterized as lethal synthesis, resulting in reduced activity of pathogenic microorganisms and improved conditions of oral cavity organs. Xylitol facilitates transition of biologically active components into teeth tissues. (Tanzer J. M./Xylitol chewing gum and dental caries.//Int. Dent. J., 1995 Feb.; 45 (1 Suppl 1):65-76).

Calcium glycerophosphate is a source of active phosphorus and calcium supply to teeth and parodontium tissues and facilitates physiological process of mineralization in enamel/saliva system. It improves anticarious effect of formulation and remineralization in non-carious teeth affections. Furthermore, it intensifies anabolic processes in tissues that is important for teeth disease prophylaxis.

Magnesium is a structural component of teeth. Magnesium is incorporated in complex formulation as microelement, which is a cofactor for phosphatase. Under the influence of these enzymes, incorporation of phosphates into solid teeth tissues increases.

There is added guar gum to formulation for the increase of teeth film consistency, containing mineral additives, and for the increase of adhesion of this film to solid tissues as well. Guar gum powder is produced out of endosperm of seeds Cyamopsis tetraganoloba—a plant, known as guar. According to its chemical composition guar gum is a non-ionique polysaccaride of vegetable origin. Molecular structure is a direct chain, formed by galactose and mannose, therefore, guar gum is galactomannan, with mannose-galactose ratio of about 2:1. If guar gum is dissolved in cold or hot water, it forms high-viscosity gel (Industrial Gums. Third edition: edited by Whistler R. L. and BeMiller J. N.; Academic Press 1993).

At best invention realization presupposes that gel contains the following as inert components, wt %

| | |
|---|---|
| Glycerine | 2-20 |
| Sorbitol | 2-20 |
| Hydroxyethyl cellulose | 1.5-3.0 |
| Non-ionic surface-active component | 0.2-1.5 |
| Preservative | 0.01-0.30 |
| Flavor | 0.02-0.50 |
| Drinking water | The rest |

In the process of gel preparation as non-ionic surface-active components there can be utilized polysorbate-20, PEG-40 hydrogenated castor oil (polyoxyethylene (40) hydrogenated castor oil), alkylpolyglucoside.

As preservatives there can be used such components as methylparaben, propylparaben, or their sodium salts as well such as phenoxyethanol, benzole acid, sodium benzoate, potassium sorbate, thymol.

Thus, there is invented high-efficiency formulation in the form of gel which is used after cleaning of soft dental deposit day by day in a series of 2-4 weeks tests depending on clinical situation in oral cavity for caries prophylaxis, non-carious affections, teeth hypersensitivity treatment and for improvement of teeth visual appearance (color and gloss).

### REALIZATION OF INVENTION

The possibility of invention realization is confirmed by examples of specific formulations. The ratio of components is demonstrated in Table 1.

Formulations are prepared in the following way.

Heat the required quantity of water in a measuring bin up to 75-80° C., then feed heated water into mixer.

Add methylparaben, magnesium chloride (or magnesium sulfate in examples 3 and 4), xylitol, sorbitol.

Mix for 20 minutes till formation of transparent solution.

Prepare separately suspension of hydroxyethyl cellulose, guar gum and calcium glycerophosphate in glycerine.

Cool water solution to 50-55° C., add suspension and mix for 30 minutes till formation of homogeneous mass.

Heat polysorbate-20 (or PEG-40 hydrogenated castor oil in examples 1 and 4) separately to 45-50° C. Add flavor and stir for 10 minutes till formation of homogeneous mix.

Add this mix to the mass obtained before and mix for 20-30 minutes till formation of homogeneous gel.

Cool the obtained gel to 20-25° C. in the process of mixing and pack it into tubes made of polymer material.

TABLE 1

| | Concentration, % wt. | | | |
|---|---|---|---|---|
| | Formu- | Formu- | Formu- | Formu- |
| Components | lation 1 | lation 2 | lation 3 | lation 4 |
| Glycerine | 2 | 8 | 15 | 20 |
| Sorbitol | 20 | 15 | 10 | 2 |
| Xylitol | 2 | 10 | 15 | 20 |
| Methylparaben | 0.1 | 0.2 | 0.24 | 0.3 |
| Guar gum | 0.3 | 0.2 | 0.06 | 0.03 |
| Hydroxyethyl cellulose | 3 | 2.5 | 1.9 | 1.5 |
| Flavor | 0.5 | 0.2 | 0.08 | 0.01 |
| Calcium glycerophosphate | 0.1 | 0.7 | 1.5 | 3 |
| Magnesium chloride | 0.01 | 0.06 | — | — |
| Magnesium sulfate | — | — | 0.2 | 0.5 |
| Polysorbate-20 | — | 0.9 | 0.5 | — |
| PEG-40 hydrogenated castor oil | 1.5 | — | — | 0.2 |
| Drinking water | Up to | Up to | Up to | Up to |
| | 100% | 100% | 100% | 100% |

Method of manufacturing and sequence of operations are equal in all formulation examples as also according to patent claims they are the same for other cases which the invention covers.

Application technique.

Application can be carried out in two ways:

using standard tray

without using tray, with cotton swabs or without them

For intense mineralization (in cases of acute caries, erosion and other non-carious affections treatment) a thin flat layer of gel is applied on teeth by a brush or any other means and then covered by tray. Application takes 12-15 minutes, then gel gluts are expectorated. After application a patient should neither have any meal or drink, nor rinse the mouth for an hour, which provides for an extended effect.

Teeth application without tray is carried out in the following way: soft tissues of cheeks, lips and tongue are isolated by cotton swabs, a thin flat layer of formulation is applied on teeth for 7-12 minutes. After application cotton swabs and gel gluts are expectorated. It is not recommended to have any meal or drink and rinse cavity for an hour.

### EXAMPLES OF ADMINISTERED USE OF FORMULATION

### Example 1

Patient X, 38 years of age, complained on hypersensitivity and unsatisfactory cosmetic teeth appearance.

During the first examination on the 4th of April 2005 it was established:

HI (hygiene index)*⁾—1.95 points *⁾—Yu. A. Fedorov, V. A. Drozhzhina, Clinical course, diagnostics and treatment of noncarious lesions of teeth. Novoe v stomatologii, No 10 (60), 1997, Special Issue.).

ITHP*⁾—54%

ITHI*⁾—2.8 points

Teeth color according to Vita scale—C3, gloss was not enough

A four-week course of formulation application with isolating swabs was administered to the patient.

The next examination was carried out in 4 weeks on the 3d of May 2005. There were no complaints.

Objective data:

HI (hygiene index)—1.57 points

ITHP—6.2%

ITHI—0

Teeth color according to Vita scale—C1, gloss became more intense.

### Example 2

Patient Y, 28 years of age, complained on teeth hypersensitivity, partial absence of gloss and unsatisfactory teeth color on the 9th of June 2005

Objective data:

HI—2.17 points

ITHP—62.5%

ITHI—2.1 points

Teeth color could not be determined, but there was noted prevalence of yellow tones over white tones.

It was recommended to do formulation applications with standard tray for 3 weeks day by day.

The next examination was conducted in 3 weeks on the 8th of July 2005. There were no complaints, the patient noted insignificant reaction on very cold meal (ice cream).

Objective data

HI (hygiene index)—1.5 points

ITHP—9.3%

ITHI—1.1 points

Teeth whitening was visually noticed, teeth gloss became more intense which satisfied the patient.

### Example 3

Patient Z, 36 years of age, complained on teeth hypersensitivity after a course of home bleaching. During the first examination on the 2d of August 2005 it was determined that:

Teeth color according to Vita-scale—C2, gloss was not enough

HI—1.9 points

ITHI—2.1 points

ITHP—62.5%

It was recommended to do formulation applications with standard tray for four weeks day by day. Next examination was in four weeks on the 31st of August 2005. No complaints.

Teeth color according to Vita scale—A1, teeth gloss became more intense

ITHP, ITHI—0

HI—1.2 points

The results summed up after observations of remineralizing gel effect have been correlated with the data obtained in experiments with fluorine-containing tooth pastes and gels used more frequently for these purposes in modern practice.

Main group (remineralizing gel)

Correlation group (fluorine-containing tooth pastes and gels)

Comparison results are presented in table 2.

Besides 89.7% of patients examined have positively evaluated teeth color (teeth have become whiter) and enamel gloss has improved which reveals active remineralizing effect of formulation.

TABLE 2

| | | | Fluoride formulations | |
|---|---|---|---|---|
| | The offered formulation | | (commercial samples) | |
| | initial | In 4 weeks | initial | In 4 weeks |
| Hygiene Index (points) | 2.12 ± 0.13 | 1.5 ± 0.12 | 2.30 ± 0.21 | 1.79 ± 0.18 |
| ITHP (index of teeth | 39.5 ± 3.3 | 9.3 ± 0.6 | 33.0 ± 1.9 | 19.3 ± 0.9 |
| hypersensitivity prevalence) % | | | | |
| ITHI (index of teeth | 21.0 ± 0.9 | 1.01 ± 0.05 | 2.3 ± 0.7 | 1.61 ± 0.8 |
| hypersensitivity intensity) | | | | |
| points | | | | |

1. Gel for teeth tissues remineralization comprising xylitol 2-25 wt. %, calcium glycerophosphate 0.1-3.0 wt. %, magnesium ion source 0.01-0.50 wt. % and guar gum 0.03-0.30 wt. % as active components and inert components as well used for teeth gels preparation, herein as magnesium ion source there is used one of the components sorted out of the group containing magnesium chloride, magnesium sulfate or magnesium nitrate.

2. Gel according to claim 1, characterized by containing the following components as inert, wt. %:

| | |
|---|---|
| Glycerine | 2-20 |
| Sorbitol | 2-20 |
| Hydroxyethyl cellulose | 1.5-3.0 |
| Non-ionic surface-active component | 0.2-1.5 |
| Preservative | 0.01-0.30 |
| Flavor | 0.02-0.50 |
| Drinking water | The rest |

3. Gel according to claim 2 characterized by containing as non-ionic surface-active component one of the components sorted out of the group: polysorbate-20, PEG-40 hydrogenated castor oil, alkylpolyglucoside.

4. Gel according to claim 2 characterized by containing as preservative one of the components sorted out of the group: methylparaben, propylparaben, or their sodium salts as well as phenoxyethanol, benzole acid, sodium benzoate, potassium sorbate, thymol.
